(19) United States
(12) Patent Application Publication
(10) Pub. No.: US 2025/0228925 A1
(43) Pub. Date: Jul. 17, 2025

(54) METHODS AND RELATED ASPECTS FOR INCREASING ANTIGENIC INSERTION SITES ON A RECOMBINANT IMMUNE COMPLEX PLATFORM

(71) Applicant: ARIZONA BOARD OF REGENTS ON BEHALF OF ARIZONA STATE UNIVERSITY, Scottsdale, AZ (US)

(72) Inventors: Hugh Mason, Phoenix, AZ (US); Mary Pardhe, Phoenix, AZ (US); Andrew Diamos, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/698,971

(22) PCT Filed: Oct. 19, 2023
(86) PCT No.: PCT/US2023/077320

§ 371 Date: Apr. 5, 2024

Related U.S. Application Data
(60) Provisional application No. 63/380,076, filed on Oct. 19, 2022.

(51) Int. Cl. A61K 39/12; A61K 39/00; C07K 14/005; C12N 7/00; C12N 15/85

(52) U.S. Cl. CPC A61K 39/12; C07K 14/005; C12N 7/00; C12N 15/85; A61K 2039/70; C07K 2319/30; C07K 2319/40; C12N 2770/24122; C12N 2770/24134

(57) ABSTRACT

Provided herein is a multivalent recombinant immune complex (RIC) that includes an immunoglobulin heavy chain; at least one epitope tag, wherein the immunoglobulin heavy chain binds the epitope tag; and at least fragments of antigens from at two viral serotypes. In some embodiments, the antigens comprise a dengue virus (DENV) domain III antigen. Related methods and other aspects are also provided.

### CROSS-REFERENCE TO RELATED APPLICATIONS

This application claims the benefit of U.S. Provisional Patent Application Ser. No. 63/380,076, filed Oct. 19, 2022, the disclosure of which is incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on Oct. 19, 2023, is named “0391.0021-PCT.xml” and is 38,482 bytes in size. The sequence listing contained in this .XML file is part of the specification and is hereby incorporated by reference herein in its entirety.

### BACKGROUND

Recombinant immune complexes (RICs), fundamentally, are composed of immunoglobulin molecules specific for a desired antigen that are fused to the same antigen that the antibody is specific for. Specifically, the parts of an RIC are an antibody, linked via its C-terminus, to an antigen that is followed by an epitope tag for the antibody. This allows for the binding region of one antibody to bind to the antigen recombinantly fused to another antibody, resulting in the formation of large, highly immunogenic antibody-antigen complexes. RICs can be engineered into ‘universal vaccine platforms’ through the use of antibodies specific for an epitope tag, which allows for the same antibody to be used regardless of the antigen so long as the antibody's corresponding epitope tag is expressed on the antigen (FIG. 1). Thus, RIC can potentiate the immunogenicity of a given antigen.

A serotype or serovar is a distinct variation within a species of bacteria or virus or among immune cells of different individuals. In the case of dengue virus (DENV), for example, there are four serotypes that circulate in humans and immunogenically cross-react with each other to produce antibody-dependent enhancement. The presence of this antibody-dependent enhancement makes it important for vaccine candidates to provide a balanced immune response against all four serotypes.

Accordingly, there is a need for vaccination compositions of use in generating an immune response against multiple serotypes of a given microorganism, such as a pathogenic viral agent.

### SUMMARY

The present disclosure relates, in certain aspects, to vaccination compositions that comprise multivalent recombinant immune complexes (RICs) effective at generating immune responses against multiple viral serotypes in mammalian subjects. The present disclosure also provides methods of producing the vaccination compositions in addition to methods of generating an immune response against HSV2 in a mammalian subject. These and other aspects will be apparent upon a complete review of the present disclosure, including the accompanying figures.

In one aspect, the present disclosure provides a vaccination composition that comprises a multivalent recombinant immune complex (RIC), comprising: an immunoglobulin heavy chain; at least one epitope tag, wherein the immunoglobulin heavy chain binds the epitope tag; and at least fragments of antigens from at two viral serotypes.

In some embodiments, the antigens are from at least three viral serotypes, at least four viral serotypes, at least five viral serotypes, at least six viral serotypes, or more viral serotypes. In some embodiments, the antigens comprise a dengue virus (DENV) domain III antigen from four DENV serotypes. In some embodiments, the epitope tag is linked to the antigens. In some embodiments, the epitope tag is linked to the C-terminus and/or the N-terminus of the immunoglobulin heavy chain. In some embodiments, one or more of the antigens are linked to the C-terminus and/or the N-terminus of the immunoglobulin heavy chain. In some embodiments, the multivalent RIC further includes an immunoglobulin light chain. In some embodiments, the multivalent RIC includes an Immunoglobulin G (IgG). In some embodiments, the multivalent RIC includes a human or humanized IgG.

In one aspect, the present disclosure provides a method of generating an immune response against a virus in a mammalian subject, the method comprising: administering to the mammalian subject a multivalent recombinant immune complex (RIC) that comprises: an immunoglobulin heavy chain; at least one epitope tag, wherein the immunoglobulin heavy chain binds the epitope tag; and at least fragments of antigens from at two viral serotypes.

In some embodiments, the virus comprises a dengue virus (DENV) and wherein the antigens comprise a DENV domain III antigen from four DENV serotypes. In some embodiments, the antigens comprise a dengue virus (DENV) domain III antigen from four DENV serotypes. In some embodiments, the epitope tag is linked to the antigens. In some embodiments, the epitope tag is linked to the C-terminus and/or the N-terminus of the immunoglobulin heavy chain. In some embodiments, one or more of the antigens are linked to the C-terminus and/or the N-terminus of the immunoglobulin heavy chain. In some embodiments, the RIC further comprises an immunoglobulin light chain. In some embodiments, the RIC comprises an Immunoglobulin G (IgG). In some embodiments, the IgG comprises a human or humanized IgG.

In one aspect, the present disclosure provides a recombinant vector comprising a nucleic acid molecule that encodes a recombinant immune complex (RIC), wherein the RIC comprises an immunoglobulin heavy chain; at least one epitope tag, wherein the immunoglobulin heavy chain binds the epitope tag; and at least fragments of antigens from at least two viral serotypes. In some embodiments, a plasmid comprises the recombinant vector.

### BRIEF DESCRIPTION OF THE DRAWINGS

The accompanying drawings, which are incorporated in and constitute a part of this specification, illustrate certain embodiments, and together with the written description, serve to explain certain principles of the compositions, methods, and related aspects disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1 depicts an exemplary diagram of universal recombinant immune complex (RIC) targeting an antigen. In this example, the antigen is linked to the C-terminus of the immunoglobulin heavy chain. The epitope tag of one immunoglobulin is bound to the binding site of other immunoglobulins, forming complexes that contain the target antigen.

FIGS. 2A-2C: Construct depictions and expression analysis of DV1-4 RICs (A) Schematic depictions of the constructs used in this example. The stars indicate the serotype of the DV antigen while the circle symbolizes the shortened self-reactive antibody tag. The murine antibody shown as DV1 mRIC represents the different species of antibody. (B) A leaf expressing DV1-4 C-RIC and DV1-4 N-RIC and harvested 5 DPI. (C) A Western blot containing crude plant extract samples of DV1-4 C-RIC or DV1-4 N-RIC. The membrane was probed with an anti-human IgG H+L+HRP probe.

FIGS. 3A-3C: Expression analysis of individual DV RICs and DV 2H2 (A) A leaf expressing the four individual DV N-RICs that was harvested at 4 DPI. (B). A Western blot of crude plant extract samples from each individual DV RIC. The samples were detected with an anti-human IgG (H+L)+HRP probe. (C) SDS-PAGE gel containing purified samples of the DV1 mRIC, four individual DV N-RICs, DV 2H2, DV1-4 N-RIC and DV1-4 C-RIC.

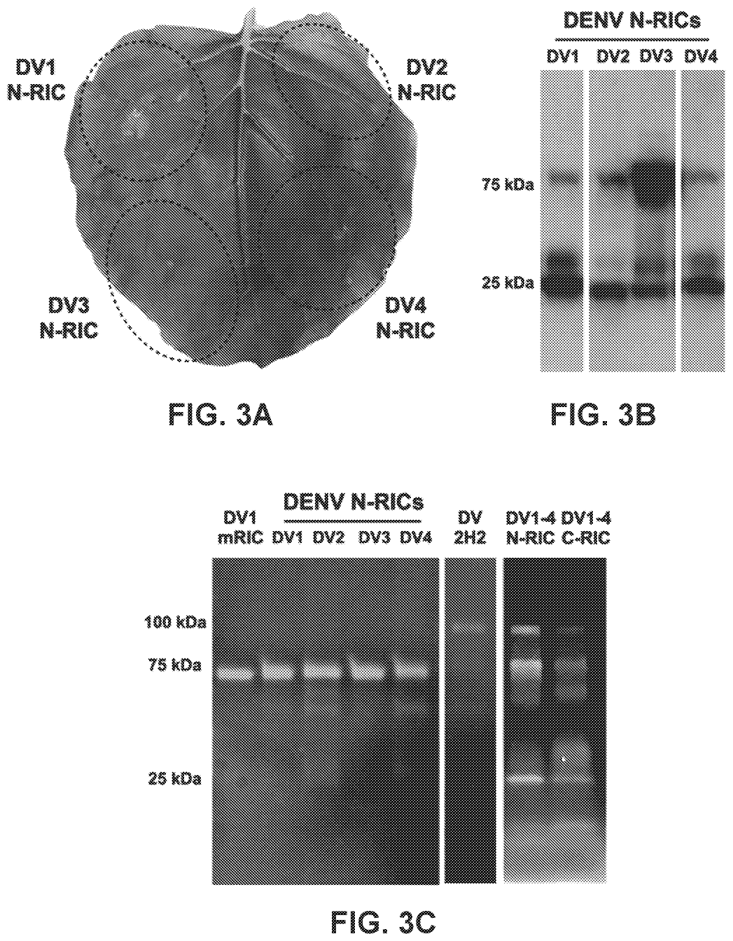

FIGS. 4A and 4B: C1q and FcγRIII binding assay (A) Human complement C1q receptor was bound to polystyrene plates and probed with 0.1 mg/ml of purified DENV constructs and a 10-fold dilution thereof. The samples containing a human antibody were detected with an anti-human IgG (H+L)+HRP probe while the mouse samples were detected with an anti-mouse IgG (H+L)+HRP probe. (B) Human FcγRIIIA/CD16a receptor was probed with equimolar concentrations of purified DV sample and detected with species-specific, HRP-conjugated antibodies.

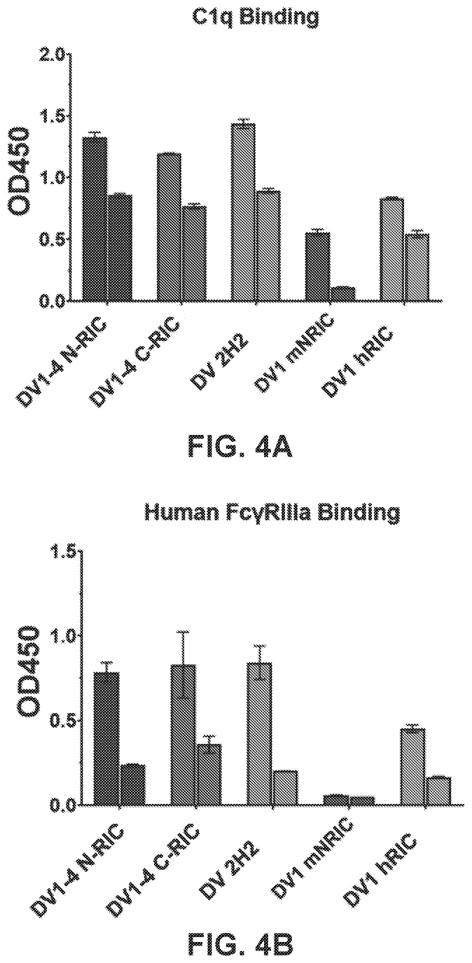

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., a vaccination composition) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antibody: As used herein, the term “antibody” refers to an Immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a viral protein, e.g., a molecule in which one or more immunoglobulin chains is not full length, but which specifically binds to the protein. Examples of binding portions encompassed within the term “antigen-binding portion of an antibody include (i) a Fab fragment, a monovalent fragment consisting of the VLC, VHC, CL and CH1 domains: (ii) a F(ab′)2 fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV). Such single chain antibodies are also encompassed within the term “antigen binding portion” of an antibody. These antibody portions are obtained using conventional techniques known to those with skill in the art, and the portions are screened for utility in the same manner as are Intact antibodies.

Epitope: As used herein, “epitope” refers to the part of an antigen to which an antibody and/or an antigen binding portion binds.

Immune complex. As used herein, the term “immune complex” refers to a complex comprising immunoglobulin molecules or fragments thereof bound to its cognate antigen. As used herein, the term “recombinant immune complex” or “RIC” refers to an immune complex that is not produced by the species that originally produces the immunoglobulin molecule in the immune complex. For example, an exemplary recombinant immune complex comprises human immunoglobulin but is synthesized by plants.

Immune response: As used herein, the term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An Immune response includes, but is not limited to, an innate and/or adaptive immune response. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

Immunogen: As used herein, the term “immunogen” or “immunogenic” refers to a compound, composition, or substance which is capable, under appropriate conditions, of stimulating an immune response, such as the production of antibodies or a T cell response in an animal, including compositions that are injected or absorbed into an animal. As used herein, “immunize” means to render a subject protected from an infectious disease.

Subject: As used herein, “subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

Vaccination: As used herein, the term “vaccination” or “vaccinate” refers to the administration of a composition intended to generate an immune response, for example to a disease-causing agent such as a pathogenic virus. Vaccination can be administered before, during, and/or after exposure to a disease-causing agent, and/or to the development of one or more symptoms, and in some embodiments, before, during, and/or shortly after exposure to the agent. Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, or intramuscular. Vaccines may be administered with an adjuvant to boost the immune response. In some embodiments, vaccination includes multiple administrations, appropriately spaced in time, of a vaccinating composition.

### DETAILED DESCRIPTION

The present disclosure relates, in certain aspects, to multivalent recombinant immune complexes (RICs) of use in generating an immune response against multiple viral serotypes. The immunogenicity of an immune complex (IC) typically depends strongly on the individual properties of each antibody and the oligomeric nature of its cognate antigen. Therefore, recombinant immune complex (RIC) systems were created which contain well-characterized human IgG1 tagged with its own binding site, allowing self-multimerizing immune complexes to be formed with any antigen on a single universal platform. Additional details concerning RIC systems and related aspects are also described in, for example, U.S. patent application Ser. No. 16/404,698, filed May 6, 2019, Ser. No. 16/976,739, filed Aug. 28, 2020, and Ser. No. 17/190,745, filed Mar. 3, 2021, which are each incorporated by references in their entirety.

In some embodiments, dengue virus (DENV) envelope domain III antigen is used in the multivalent RICs disclosed herein. To improve the Immunogenicity of the domain III antigen, researchers have explored a multitude of fusion strategies including fusing the domain III antigen to IgG-based scaffolds, virus-like particles, Toll-like receptors or other immunostimulatory molecules. There are four DENV serotypes that circulate in humans and immunogenically cross-react with each other to produce antibody-dependent enhancement. The presence of this antibody-dependent enhancement makes it important for vaccine candidates to provide a balanced immune response against all four serotypes. In the examples presented herein, three RIC variants were created that displayed four antigens in various fusion arrangements and compared their expression levels, immunogenicity, and ability to bind the complement C1q receptor and FcγRII receptor. By using the DENV domain III antigen from the four Dengue serotypes, the inventors were able to control for size in the various fusion arrangements. In addition, in these examples, the immunogenicity was compared between a RIC containing either a mouse antibody or human antibody to assess whether the antibody species played a role in the immunogenicity of the RIC construct when tested in mouse immunization studies.

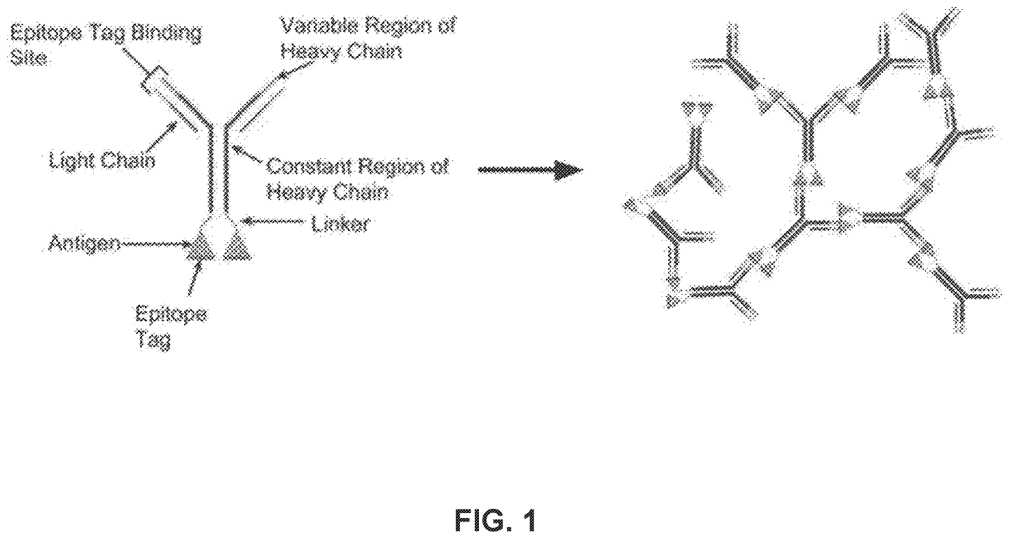

In some embodiments, the RICs described herein comprise an immunoglobulin heavy chain, an epitope tag that can bind to the immunoglobulin heavy chain, and a target antigen. In some aspects, the immunoglobulin heavy chain is a camelid immunoglobulin. In certain embodiments, the RIC further comprises an immunoglobulin light chain. Thus, in some aspects, the RIC comprises a standard antibody (two heavy chains and two light chains joined to form a “Y” shaped molecule), an antigen, and an epitope tag that is recognized by the antibody (FIG. 1). The antibody binds to the epitope tags on other antibody fusions and forms a complex. In some embodiments, the RIC comprises human IgG, and the epitope tag is a 6H epitope tag.

RICs described herein include conventional RICs where the target antigen is linked to the C-terminus of the immunoglobulin heavy chain and the epitope tag is linked to the other end of the target antigen (also referred to herein as “C-RIC”). The recombinant immune complex is produced by fusing a target antigen to the C-terminus of the heavy chain of an immunoglobulin that binds specifically to the antigen, wherein the co-expression of this fusion protein with the light chain of the antibody produces a fully formed immunoglobulin that is self-reactive, and results in the creation of an immune complex due to the bivalent binding capacity of the immunoglobulin. However, antigens with inaccessible N-termini cannot be easily used in the RIC platform without disrupting native antigenic conformation. Also described herein is a novel design of RIC where the target antigen is linked to the N-terminus of the immunoglobulin heavy chain and the epitope tag is linked to C-terminus of the immunoglobulin heavy chain (also referred to herein as “N-RIC”). In some embodiments, the RICs of the present disclosure are co-administered to subjects along with virus-like particles (VLPs) to produce a greater immune response than can be obtained through delivering either alone at the same does of the antigen.

In certain embodiment of an expression vector encoding RICs, the expression vector comprises a expression cassette encoding the immunoglobulin heavy chain, the target antigen, and the epitope tag. In some aspects, the expression vector further comprises a second expression cassette encoding the immunoglobulin light chain. These and other attributes of the present disclosure will be apparent upon a complete review of the specification, including the accompanying figures.

In some embodiments, a multivalent recombinant RIC or a component thereof of the present disclosure is encoded by a synthetic polynucleotide that comprises one or more of the nucleotide sequences (or complements thereof) of SEQ ID. NOS: 1-15 (shown below in Table 1) or comprises a polynucleotide having at least 80%, 85%, 90%, 95%, 99% sequence identity with one or more of SEQ ID NOS: 1-15. The phrase “functional portion, fragment, or variant thereof” in the context of the proteins described herein, refers to a portion or fragment of the full-length protein or a non-wild-type form of the protein (full-length, portion, or fragment thereof) that retains a desired property or function, such as improving immunogenicity, effectuating vaccination, or the like. In some embodiments, vaccine compositions of the present disclosure comprise RICs that comprise dengue virus (DENV) domain III proteins, or functional portions, or fragments or variants thereof from multiple DENV serotypes.

TABLE 1

| | SEQ | |
|---|---|---|
| | ID | |
| DNA SEQUENCE | NO | Description |
| ATGGGATGGTCTTGCATCATACTCTTTCTTGTTGCAACTGCTACAGGTGTCCACTCT | 1 | IgG heavy |
| | | chain |
| | | signal |
| | | peptide |
| GATGTTCAGCTTCTCGAGTCTGGAGGTGGTCTTGTGCAACCTGGAGGTTCCTTGAGACTCTC | 2 | IgG1 heavy |
| CTGTGCAGCTTCAGGGTTTGACTTCAGTAGGTACTGGATGAGTTGGGTTCGTCAAGCTCCTG | | chain |
| GGAAAGGACTAGAATGGATTGGAGAGATCAATCCAGATTCAAGTACCATCAACTATACTCCAT | | |
| CTCTGAAGGATCGCTTCACCATTTCCAGAGACAATGCCAAGAACACGTTGTATCTTCAGATGA | | |
| ACAGCTTGAGGACTGAAGACACAGCCTTGTACTACTGCACAAGACAGGGCTATGGCTACAAC | | |
| TACTGGGGTCAAGGCACCACTGTCACAGTGTCTTCAGCTAGCACCAAAGGTCCATCGGTCTT | | |
| TCCACTGGCACCTTCTTCCAAGAGTACTTCTGGAGGCACAGCTGCACTGGGTTGTCTTGTCA | | |
| AGGACTACTTTCCAGAACCTGTTACGGTTTCGTGGAACTCAGGTGCTCTGACCAGTGGAGTGC | | |
| ACACCTTTCCAGCTGTTCTTCAGTCCTCAGGATTGTATTCTCTTAGCAGTGTTGTGACTGTTC | | |
| CATCCTCAAGCTTGGGCACTCAGACCTACATCTGCAATGTGAATCACAAACCCAGCAACACCA | | |
| AGGTTGACAAGAAAGTTGAGCCCAAGTCTTGTGACAAGACTCATACGTGTCCACCGTGCCCA | | |
| GCACCTGAACTTCTTGGAGGACCGTCAGTCTTCTTGTTTCCTCCAAAGCCTAAGGATACCTTG | | |
| ATGATCTCCAGGACTCCTGAAGTCACATGTGTAGTTGTGGATGTGAGCCATGAAGATCCTGA | | |
| GGTGAAGTTCAACTGGTATGTGGATGGTGTGGAAGTGCACAATGCCAAGACAAAGCCGAGAG | | |
| AGGAACAGTACAACAGCACGTACAGGGTTGTCTCAGTTCTCACTGTTCTCCATCAAGATTGGT | | |
| TGAATGGCAAAGAGTACAAGTGCAAGGTCTCCAACAAAGCCCTCCCAGCCCCCATTGAGAAG | | |
| ACCATTTOCAAAGCGAAAGGGCAACCCCGTGAACCACAAGTGTACACACTTCCTCCATCTCG | | |
| CGATGAACTGACCAAGAACCAGGTCAGCTTGACTTGCCTGGTGAAAGGCTTCTATCCCTOTG | | |
| GTTCTCGATTCTGACGGCTCCTTCTTCCTCTACAGCAAGCTCACAGTGGACAAGAGCAGGTG | | |
| ACATAGCTGTAGAGTGGGAGAGCAATGGGCAACCGGAGAACAACTACAAGACTACACCTCCC | | |
| GTTCTCGATTCTGACGGCTCCTTCTTCCTCTACAGCAAGCTCACAGTGGACAAGAGCAGGTG | | |
| GCAACAAGGGAATGTCTTCTCATGCTCCGTGATGCATGAGGCTCTTCACAATCACTACACACA | | |
| GAAGAGTCTCTCCTTGTCTCCGGGTAAA | | |
| GGAGGTGGCGGATCAGGTGGAGGCGGTTCAGGCGGAGGTGGATCC | 3 | Linker |
| TCTTACGTTATGTGCACTGGATOTTTTAAGCTTGAGAAAGAGGTGGCCGAAACCCAACACGGT | 4 | DENV1 |
| ACTGTTTTGGTTCAAGTTAAGTACGAGGGTACCGACGCACCATGCAAAATCCCCTTCTCTTCT | | EdIII |
| CAAGATGAGAAGGGAGTTACTCAGAACGGTAGACTTATCACCGCAAATCCTATAGTTACAGAT | | |
| AAGGAGAAACCTGTGAATATAGAGGCCGAGCCTCCGTTCGGGGAGTCTTATATCGTTGTCGG | | |
| CGCTGGGGAGAAGGCGCTTAAATTGTCATGGTTCAAGAAGGGGTCATCA | | |
| GGAGGTTCAGGTTCC | 5 | Linker |
| | | (1-2) |
| | | DENV2 |
| TOTTACTCTATGTGCACTGGGAAATTCAAAGTCGTTAAGGAGATAGCTGAAACTCAGCACGGA | 6 | EdIII |
| ACCATCGTTATTAGAGTTCAGTACGAAGGAGATGGATCTCCATGCAAAACCCCTTTTGAAATC | | |
| ATGGACCTTGAGAAGAGACACGTTTTGGGTAGACTTACTACTGTGAACCCGATCGTTACTGAG | | |
| AAGGATTCCCCAGTGAACATAGAAGCTGAGCCTCCGTTCGGTGACTCTTACATCATCATCGGT | | |
| GTTGAGCCTGGCCAGCTCAAGTTAGACTGGTTTAAGAAGGGTTCATCC | | |
| GGAGGTTCAGGTTCT | 7 | Linker |
| | | (2-3) |
| | | DENV3 |
| TCTTACGCTATGTGTTTGAATACCTTCGTTTTGAAGAAAGAAGTTTCTGAAACCCAGCACGGA | 8 | EdIII |
| ACCATCCTTATTAAGGTTGAGTACAAGGGAGAAGACGCTCCATGCAAGATCCCTTTCTCTACT | | |
| GAGGATGGTCAAGGCAAGGCCCACAACGGGAGACTTATCACTGCAAACCCAGTCGTTACCAA | | |
| GAAAGAAGAGCCAGTAAACATCGAGGCTGAACCACCCTTTGGAGAGTCCAACATAGTGATCG | | |
| GTATCGGAGACAAGGCTCTTAAGATCAATTGGTATCGTAAAGGCTCATCA | | |
| GGAGGTTCAGGATCT | 9 | Linker |
| | | (3-4) |
| TCTTACACTATGTGTTCTGGAAAGTTTTCTATCGATAAGGAGATGGCTGAGACTCAGCACGGT | 10 | DENV4 |
| ACAACTGTTGTTAAGGTTAAGTACGAAGGAGCCGGAGCCCCATGCAAAGTTCCAATCGAGAT | | EdIII |
| TAGAGACGTCAACAAAGAGAAGGTCGTTGGAAGGATCATCTCTCCCACCCCATTTGCTGAAA | | |
| ACACCAACTCTGTTACAAACATCGAATTGGAAAGACCTCTCGACTCCTATATCGTTATCGGAG | | |
| TTGGCGATTCTGCTCTTACCCTTCACTGGTTCAGAAAGGGATCTTCT | | |
| ACTAGT | 11 | Spel linker |
| TACAAGCTGGACATATCT | 12 | Epitope tag |
| | | “d” |
| ATGGGATGGTCTTGCATCATTCTCTTCTTGGTAGCCACAGCTACAGGTGTCCACTCC | 13 | IgG light |
| | | chain |
| GATGTTTTGATGACTCAAAGCCCTCTCTCACTTCCTGTGACTCTTGGACAGCCCGCATCCATA | 14 | IgG light |
| TCTTGCAGATCTAGTCAGAGTATTGTTCATAGTAACGGCAACACCTACTTGGAATGGTATCTG | | chain kappa |
| CAGAAACCAGGCCAGTCTCCAAAGCTTCTGATCTACAAGGCTTCCAATCGTTTCTCTGGTGTC | | |
| CCAGACAGGTTTAGTGGCAGTGGATCAGGGACTGACTTCACATTGAAGATCAGCAGAGTTGA | | |
| GGCTGAAGATGCGGGAGTGTACTATTGTCTTCAAGGTTCACATGTTCCGTCAACGTTTGGAG | | |
| GTGGGACCAAAGTGGAGATCAAGACTGTTGCGGCGCCATCTGTCTTCATCTTTCCTCCATCT | | |
| GATGAACAACTCAAGTCTGGAACTGCTTCTGTTGTGTGCCTTCTGAACAACTTCTATCCTAGA | | |
| GAAGCCAAAGTACAGTGGAAGGTTGACAATGCTCTTCAATCAGGTAACTCCCAGGAGAGTGT | | |
| CACAGAGCAAGATTCCAAGGATTCCACCTACAGCCTCTCAAGTACCTTGACGTTGAGCAAGG | | |
| CAGACTATGAGAAACACAAAGTGTACGCATGCGAAGTCACTCATCAGGGCCTGTCATCACCC | | |
| GTGACAAAGAGCTTCAACAGGGGAGAGTGT | | |
| ATGGCTAACAAGCACCTCTCATTGTCTCTCTTCCTTGTGCTCCTTGGTCTTTCTGCTTCTCTT | 15 | Barley |
| GCTTCTGGT | | alpha |
| | | amylase |
| | | signal |
| | | peptide |

In some embodiments, a multivalent recombinant RIC of the present disclosure comprises a polypeptide comprising one or more of the amino acid sequences of SEQ ID. NO: 16-28 (shown below in Table 2) or comprises a polypeptide having at least 80%, 85%, 90%, 95%, 99% sequence identity with one or more of SEQ ID NOS: 16-28.

TABLE 2

| | SEQ | |
|---|---|---|
| | ID | |
| AMINO ACID SEQUENCE | NO | Description |
| MGWSCHILFLVATATGVHS | 16 | IgG heavy |
| | | chain |
| | | signal |
| | | peptide |
| DVQLLESGGGLVQPGGSLRLSCAASGFDFSRYWMSWVRQAPGKGLEWIGEINPDSSTINYTPSL | 17 | IgG heavy |
| KDRFTISRDNAKNTLYLQMNSLRTEDTALYYCTRQGYGYNYWGQGTTVTVSSASTKGPSVFPLA | | chain |
| PSSKSTSGGTAALGCLVKDYFPEPVTVSWNSGALTSGVHTFPAVLQSSGLYSLSSVVTVPSSSLG | | |
| TQTYICNVNHKPSNTKVDKKVEPKSCDKTHTCPPCPAPELLGGPSVFLFPPKPKDTLMISRTPEVT | | |
| CVVVDVSHEDPEVKFNWYVDGVEVHNAKTKPREEQYNSTYRVVSVLTVLHQDWLNGKEYKCKV | | |
| NYKTTPPVLDSDGSFFLYSKLTVDKSRWQQGNVFSCSVMHEALHNHYTQKSLSLSPGK | | |
| SNKALPAPIEKTISKAKGQPREPQVYTLPPSRDELTKNQVSLTCLVKGFYPSDIAVEWESNGQPEN | | |
| NYKTTPPVLDSDGSFFLYSKLTVDKSRWQQGNVFSCSVMHEALHNHYTQKSLSLSPGK | | |
| GGGGSGGGGSGGGGS | 18 | Linker |
| SYVMCTGSFKLEKEVAETQHGTVLVQVKYEGTDAPCKIPFSSQDEKGVTQNGRLITANPIVTDKE | 19 | DENV1 |
| KPVNIEAEPPFGESYIVVGAGEKALKLSWFKKGSS | | EdIII |
| GGSGS | 20 | Linker |
| | | (1-2, |
| | | 2-3, 3-4) |
| SYSMCTGKFKVVKEIAETQHGTIVIRVQYEGDGSPCKTPFEIMDLEKRHVLGRLTTVNPIVTEKDS | 21 | DENV2 |
| PVNIEAEPPFGDSYIIIGVEPGQLKLDWFKKGSS | | EdIII |
| SYAMCLNTFVLKKEVSETQHGTILIKVEYKGEDAPCKIPFSTEDGQGKAHNGRLITANPVVTKKEE | 22 | DENV3 |
| PVNIEAEPPFGESNIVIGIGDKALKINWYRKGSS | | EdIII |
| SYTMCSGKFSIDKEMAETQHGTTVVKVKYEGAGAPCKVPIEIRDVNKEKVVGRIISPTPFAENTNS | 23 | DENV4 |
| VTNIELERPLDSYIVIGVGDSALTLHWFRKGSS | | EdIII |
| TS | 24 | SpeI linker |
| YKLDIS | 25 | Epitope |
| | | tag “d” |
| MGWSCHILFLVATATGVHS | 26 | IgG light |
| | | chain |
| | | signal |
| | | peptide |
| DVLMTQSPLSLPVTLGQPASISCRSSQSIVHSNGNTYLEWYLOKPGQSPKLLIYKASNRFSGVPD | 27 | IgG light |
| RFSGSGSGTDFTLKISRVEAEDAGVYYCLQGSHVPSTFGGGTKVEIKTVAAPSVFIFPPSDEQLKS | | chain |
| GTASVVCLLNNFYPREAKVQWKVDNALQSGNSQESVTEQDSKDSTYSLSSTLTLSKADYEKHKV | | kappa |
| YACEVTHQGLSSPVTKSFNRGEC | | |
| MANKHLSLSLFLVLLGLSASLASG | 28 | Barley |
| | | alpha |
| | | amylase |
| | | signal |
| | | peptide |

A synthetic polynucleotide encoding a multivalent recombinant RIC, or component thereof, of the present disclosure can be comprised within an expression cassette. The term “expression cassette” or “expression vector” as used herein refers to a nucleotide sequence, which is capable of affecting expression of a protein coding sequence in a host compatible with such sequences. Expression cassettes typically include at least a promoter operably linked with the polypeptide coding sequence; and, optionally, with other sequences, e.g., transcription termination signals. Additional factors necessary or helpful in effecting expression may also be included, e.g., enhancers. “Operably linked”, refers to linkage of a promoter upstream from a DNA sequence such that the promoter mediates transcription of the DNA sequence. Thus, expression cassettes include plasmids, recombinant viruses, any form of a recombinant “naked DNA” vector, and the like. In some embodiments, expression cassettes include elements that have been codon optimized for expression in the Intended host.

The term “immunogen” or “immunogenic composition” is synonymous with “antigen or antigenic” and refers to a compound or composition comprising a peptide, polypeptide or protein which is “immunogenic,” i.e., capable of eliciting, augmenting or boosting a cellular and/or humoral immune response, either alone or in combination or linked or fused to another substance. An immunogenic composition can be a peptide of at least about 5 amino acids, a peptide of 10 amino acids in length, a fragment 15 amino acids in length, a fragment 20 amino acids in length or greater; smaller immunogens may require presence of a “carrier” polypeptide e.g., as a fusion protein, aggregate, conjugate or mixture, preferably linked (chemically or otherwise) to the immunogen. The immunogen can be recombinantly expressed from a vaccine vector, which can be naked DNA comprising the immunogen's coding sequence operably linked to a promoter, e.g., an expression cassette. The immunogen includes one or more antigenic determinants or epitopes, which may vary in size from about 3 to about 15 amino acids. In some embodiments, the immunogen or antigen is a polypeptide comprising a DENV domain III protein as described herein.

In accordance with some embodiments, the present disclosure provides a recombinant vector encoding the multivalent RIC vaccine compositions described herein. By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions, such as when a given polynucleotide encodes a functional portion, fragment, or variant of, for example, a DENV domain III protein.

Preferably, the nucleic acids of the present disclosure are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine-substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

In some embodiments, the substituted nucleic acid sequence may be optimized. Without being bound to a particular theory, it is believed that optimization of the nucleic acid sequence Increases the translation efficiency of the mRNA transcripts. Optimization of the nucleic acid sequence may involve substituting a native codon for another codon that encodes the same amino acid, but can be translated by tRNA that is more readily available within a cell, thus increasing translation efficiency. Optimization of the nucleic acid sequence may also reduce secondary mRNA structures that would interfere with translation, thus increasing translation efficiency. In some embodiments, codon optimization is performed using Genscript.

In some embodiments, the present disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleic acids of the present disclosure can be incorporated into a recombinant expression vector. In this regard, the invention provides recombinant expression vectors comprising any of the nucleic acids of the present disclosure. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally occurring as a whole. However, parts of the vectors can be naturally occurring. The recombinant expression vectors can comprise any type of nucleotide, Including, but not limited to DNA and RNA, which can be single-stranded or double-stranded, synthesized or obtained in part from natural sources, and which can contain natural, non-natural or altered nucleotides. The recombinant expression vectors can comprise naturally occurring, non-naturally occurring internucleotide linkages, or both types of linkages. Preferably, the non-naturally occurring or altered nucleotides or internucleotide linkages does not hinder the transcription or replication of the vector.

In some embodiments, the expression cassette encoding a multivalent RIC or a component thereof will be inserted into a DNA vector or plasmid. The recombinant expression vector of the present disclosure can be any suitable recombinant expression vector and can be used to transform or transfect any suitable host. Suitable vectors include those designed for propagation and expansion or for expression or both, such as plasmids and viruses. The vector can be selected from the group consisting of the pSectag2B or pVAX1 series (ThermoFisher Scientific, Carlsbad, CA), the pUC series (Fermentas Life Sciences), the pBluescript series (Stratagene, LaJolla, CA), the pET series (Novagen, Madison, WI), the pGEX series (Pharmacia Biotech, Uppsala, Sweden), pcDNA3 family of plasmids, the pNGVL4a plasmid, the pBYKEHM plasmid, the pBYKEAM plant expression vector, the BeYDV plant expression vector, and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM and pMAMneo (Clontech). The recombinant expression vectors of the present disclosure can be prepared using standard recombinant DNA techniques well known to persons having ordinary skill in the art. Constructs of expression vectors, which are circular or linear, can be prepared to contain a replication system functional in a prokaryotic or eukaryotic host cell. Replication systems can be derived, e.g., from ColEI, 2 μ plasmid, λ, SV40, bovine papilloma virus, and the like. Additional expression vectors are disclosed herein, including in an Example provided herein.

Desirably, the recombinant expression vector comprises regulatory sequences, such as transcription and translation initiation and termination codons, which are specific to the type of host (e.g., mammalian, bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected hosts. Marker genes Include biocide resistance, e.g., resistance to antibiotics, heavy metals, etc., complementation in an auxotrophic host to provide prototrophy, and the like. Suitable marker genes for the expression vectors disclosed herein may include, for instance, neomycin/G418 resistance genes, hygromycin resistance genes, histidinol resistance genes, tetracycline resistance genes, and ampicillin resistance genes, among others.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the expression vectors disclosed herein may include, for instance, neomycin/G418 resistance genes, hygromycin resistance genes, histidinol resistance genes, tetracycline resistance genes, and ampicillin resistance genes, among others.

Recombinant expression vectors can comprise a native or nonnative promoter operably linked to the nucleotide sequence encoding the multivalent RICs or components thereof, or to the nucleotide sequence which is complementary to or which hybridizes to the nucleotide sequence encoding the multivalent RICs or components thereof. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

In accordance with some embodiments, the present disclosure provides various pharmaceutical compositions comprising the multivalent recombinant RICs described herein for use as a vaccine. Thus, in further embodiments, the present disclosure provides the use of a pharmaceutical composition comprising a vaccine, and a pharmaceutically acceptable carrier, as a medicament, preferably as a medicament for the treatment of a viral infection (e.g., a DENV infection) in a subject.

In a further embodiment, the present invention provides a method for treating a viral infection (e.g., a DENV infection) in a subject in need thereof comprising administering to the subject an effective amount of the vaccine compositions described herein.

In some embodiments, the present disclosure provides methods of providing prophylaxis to, and/or treating a viral infection (e.g., a DENV infection) in, a subject in need thereof comprising administering to the subject an effective amount of a composition disclosed herein. In some embodiments, the composition is administered to the subject prior to, concurrent with, and/or after administering at least one antiviral agent to the subject. In some embodiments, the composition is administered as one or more boost doses after an initial administration of the composition to the subject.

In some embodiments, the term “administering” means that the compositions of the present disclosure are introduced into a subject, preferably a subject receiving treatment for a viral infection (e.g., a DENV infection), and the compounds are allowed to come in contact with the one or more infected cells or population of cells in vivo. In some embodiments, the composition is administered intramuscularly and/or intranasally to the subject.

It will be understood to persons having ordinary skill in the art that the vaccine compositions described herein can be administered in a regimen where there is a first or priming dose of vaccine composition administered to the subject, then after a period of time (e.g., 5 to 180 or more days), a second, third or more boost dose of vaccine is then administered to the subject. In some embodiments, the boost dose is administered 5, 6, 7, 8, 9, 10, 15, 20, 30, 40 up to 50 days apart.

In some embodiments, the carrier is a pharmaceutically acceptable carrier. With respect to pharmaceutical compositions, the carrier can be any of those conventionally used and is limited only by chemico physical considerations, such as solubility and lack of reactivity with the active compound(s), and by the route of administration. The pharmaceutically acceptable carriers described herein, for example, vehicles, adjuvants, excipients, and diluents, are well known to those skilled in the art and are readily available to the public. It is preferred that the pharmaceutically acceptable carrier be one which is chemically inert to the active agent(s) and one which has no detrimental side effects or toxicity under the conditions of use. In some embodiments, the pharmaceutical compositions of the present disclosure further include at least one additional biologically active agent (e.g., an antibiotic agent or the like). In some embodiments, the pharmaceutical compositions of the present disclosure lack a pharmaceutically acceptable carrier.

The choice of carrier will be determined in part by the chemical properties of the vaccines as well as by the particular method used to administer the vaccines. Accordingly, there are a variety of suitable formulations of the pharmaceutical composition of the invention. The following formulations for intranasal, parenteral, subcutaneous, intravenous, intramuscular, intradermal, intraarterial, Intrathecal and intraperitoneal administration are exemplary and are in no way limiting. More than one route can be used to administer the first and second vaccine, and in certain instances, a particular route can provide an immediate and more effective response than another route.

Injectable formulations are in accordance with the present disclosure according to some embodiments. Formulations for effective pharmaceutical carriers for injectable compositions are well-known to persons having ordinary skill in the art (see, e.g., Pharmaceutics and Pharmacy Practice, J.B. Lippincott Company, Philadelphia, PA, Banker and Chalmers, eds., pages 238 250 (1982), and ASHP Handbook on Injectable Drugs, Trissel, 14th ed., (2007).

In accordance with some embodiments, the vaccines of the present invention can be administered other ways known in the art. For example, the vaccines can be administered via use of electroporation techniques. Suitable electroporation techniques are disclosed in U.S. Pat. Nos. 6,010,613, 6,603,998, and 6,713,291, all of which are incorporated herein by reference. Other physical approaches can include needle-free injection systems (NFIS) (e.g., as disclosed in U.S. Pat. No. 9,333,300, which is incorporated herein by reference), gene gun, biojector, ultrasound, and hydrodynamic delivery, all of which employ a physical force that permeates the cell membrane and facilitates intracellular gene transfer. Chemical vaccination approaches typically use synthetic or naturally occurring compounds (e.g., cationic lipids, cationic polymers, lipid-polymer hybrid systems) as carriers to deliver the nucleic acid into the cells.

In some aspects the vaccines disclosed herein are formulated in a lipid nanoparticle (LNP). The use of LNPs enables the effective delivery of chemically vaccines. Both modified and unmodified LNP formulated vaccines are optionally utilized. In some embodiments the vaccines disclosed herein are superior to conventional vaccines by a factor of at least 10 fold, 20 fold, 40 fold, 50 fold, 100 fold, 500 fold or 1,000 fold.

In one set of embodiments, lipid nanoparticles (LNPs) are provided. In one embodiment, a lipid nanoparticle comprises lipids including an ionizable lipid (such as an ionizable cationic lipid), a structural lipid, a phospholipid, and the multivalent recombinant RIC vaccine. Each of the LNPs described herein or otherwise known to persons having ordinary skill in the art may be used as a formulation for the vaccines described herein. In some embodiments, the LNP comprises an ionizable lipid, a PEG-modified lipid, a phospholipid and a structural lipid. In some embodiments, the LNP has a molar ratio of about 20-60% ionizable lipid:about 5-25% phospholipid:about 25-55% structural lipid; and about 0.5-15% PEG-modified lipid. In some embodiments, the LNP comprises a molar ratio of about 50% ionizable lipid, about 1.5% PEG-modified lipid, about 38.5% structural lipid and about 10% phospholipid. In some embodiments, the LNP comprises a molar ratio of about 55% ionizable lipid, about 2.5% PEG lipid, about 32.5% structural lipid and about 10% phospholipid. In some embodiments, the ionizable lipid is an ionizable amino or cationic lipid and the phospholipid is a neutral lipid, and the structural lipid is a cholesterol. In some embodiments, the LNP has a molar ratio of 50:38.5:10:1.5 of ionizable lipid:cholesterol:DSPC:PEG2000-DMG. Additional details regarding LNPs and other carriers that are optionally adapted for use with the vaccines of the present disclosure are also described in, for example, U.S. Patent Application Publication No. U.S. 20200254086, which is incorporated by reference in its entirety.

For purposes of the present disclosure, the amount or dose of the vaccine administered should be sufficient to effect, e.g., a therapeutic or prophylactic response, in the subject over a selected time frame. The dose will typically be determined by the efficacy of the first and second vaccine and the condition of the given subject, as well as the body weight of that subject to be treated.

Typically, the attending physician will decide the dosage of first and second vaccine with which to treat each individual patient, taking into consideration a variety of factors, such as age, body weight, general health, diet, sex, to be administered, route of administration, and the severity of the condition being treated. By way of example and not intending to limit the invention, the dose of the vaccine is about 1 to 10,000 ug of vaccine to the subject being treated. In some embodiments, the dosage range of the vaccine is about 500 μg-6,000 μg of vaccine. In one exemplary embodiment, the dosage of the vaccine is about 3,000 μg.

### Example: Increasing the Antigenic Insertion Sites on the Recombinant Immune Complex Platform

### 1 Introduction

Recombinant protein subunit vaccines have several benefits that make them a promising vaccine strategy. Since they do not contain infectious material, subunit vaccines have an improved safety profile when compared to live attenuated or inactivated vaccines. Furthermore, advances in the development of various protein expression systems permit the convenient production of heterologous proteins. One challenge of subunit vaccines, though, is that they often require an adjuvant to produce an effective immune response. Many strategies have been developed to increase the immunogenicity of subunit vaccines. These include displaying the antigen on a virus-like particle structure or fusing it to a molecule, such as a toxoid, that can confer an adjuvant effect to the linked antigen.

The immunogenicity of a subunit vaccine can be enhanced via a fusion to an IgG-based protein scaffold. Polymeric IgG-fusions, in particular, have been shown to efficiently enhance the immunogenicity of various subunit antigens. This is due, in part, to the ability of polymeric IgG-fusions to effectively bind to and cross-link Fc receptors and complement receptor C1q. This activation leads to enhanced antigen uptake and presentation by dendritic cells and efficient activation of T-cells. The recombinant immune complex (RIC) platform is an IgG-based scaffold that displays an antigen fused via an N-terminal or C-terminal fusion to a well-characterized antibody that is tagged with its own epitope. When in vivo or in vitro, the RIC antibody binds to the epitope tag on surrounding RIC molecules to form highly immunogenic clusters that are capable of efficiently binding to the complement C1q receptor and FcγRIII receptor. Past work on the RIC platform has demonstrated its viability to enhance the immunogenicity of subunit antigens; however, the platform has only been tested with a single antigen fusion site located on either the antibody N-terminus or C-terminus. Increasing the number of antigenic fusion sites would greatly improve the versatility of the RIC platform by allowing for the generation of vaccine candidates that contain target multiple antigens on the same pathogen or target multiple pathogenic serotypes.

For this example, the Dengue virus (DENV) envelope domain III antigen was selected as a model antigen. The DENV envelope domain III has been a well-studied target for dengue vaccine development due to its function in cellular receptor binding and the number of neutralizing epitopes present on the protein; however, it is poorly immunogenic on its own. To improve the immunogenicity of the domain III antigen, researchers have explored a multitude of fusion strategies including fusing the domain III antigen to IgG-based scaffolds, virus-like particles, Toll-like receptors or other immunostimulatory molecules. There are four DENV serotypes that circulate in humans and immunogenically cross-react with each other to produce antibody-dependent enhancement. The presence of this antibody-dependent enhancement makes it important for vaccine candidates to provide a balanced immune response against all four serotypes. Immunization with tetravalent dengue vaccine candidates that include antigens from all four serotypes has resulted in some success and underscores the interest in developing vaccine candidates that include more than one antigen. In this example, we created three RIC variants that displayed four antigens in various fusion arrangements and compared their expression levels, immunogenicity, and ability to bind the complement C1q receptor and FcγRII receptor. By using the DENV domain III antigen from the four Dengue serotypes, we were able to control for size in the various fusion arrangements. In addition, we compared the immunogenicity between a RIC containing either a mouse antibody or human antibody to assess whether the antibody species played a role in the immunogenicity of the RIC construct when tested in mouse immunization studies.

### 2 Materials and Methods

### 2.1 Vector Construction

The antigen sequences used in this study consisted of ˜ 104 aa from domain III of four DENV serotypes (DENV-1 West Pac-74, DENV-2 PR159-S1/69, DENV-3 H87/56, DENV-4 H241-P). The DENV amino acid sequences were obtained from the GenBank entry KM229742.1 described in. These sequences were codon-optimized for expression in N. benthamiana and commercially synthesized (Integrated DNA Technologies) with appropriate primer binding sites and restriction enzyme sites. Two gBlocks® Gene Fragments were purchased, one containing domain III from DENV-1 and 2 connected by a GGSGS (named “gBlock 1-2”) (SEQ ID NO: 20) and the other containing DENV-3 and DENV-4 (named “gBlock 3-4”) connected with the same GGSGS linker (SEQ ID NO: 20).

An RIC containing all four DENV serotypes tandemly linked to the antibody C-terminus was constructed by first subcloning the four DENV serotypes into a PLITMUS28 vector, then creating the final construct. The subcloning was done with a three-fragment ligation: pLITMUS28 (New England Biolabs) was digested with SpeI-BamHI to obtain the vector fragment, gBlock 1-2 was digested BamHI-BsaI to obtain DENV-1 and 2 domain III antigens, and the gBlock 3-4 was digested with BsaI-SpeI to obtain the DENV-3 and 4 domain III antigens. The resulting construct was named pLIT-DNV114. Next, pBYKEHM-HZE3d, a construct similar to pBYR11eM-HZE3d but with the pBYKEHM vector, was digested BamHI-SpeI to obtain the vector fragment and ligated with the BamHI-SpeI insert from pLIT-DNV114 to create pBYKEHM-h6D8-DENV1t4d (“DV1-4 C-RIC”).

A RIC containing DENV-1 and 2 fused to the N-terminus of the RIC antibody was creating by digesting the first gBlock with BsaI-SpeI and ligating it into a pBYKEHM vector. The resulting construct, pBYKEHM-DV112, was then used to create a RIC containing all four DENV antigens tandemly linked to the N-terminus of the antibody. A pBYKEAM plasmid containing the RIC antibody heavy chain and short epitope tag was digested with SbfI-SpeI to obtain the vector fragment. The 5′ UTR and a portion of the tandemly linked DENV antigens were obtained with a SbfI-HindIII digest of pBYKEHM-DV112 while the remainder of the DENV antigens were obtained with a HindIII-SpeI digest of pBYKEHM-h6D8-DENV1t4d. The final construct (“DV1-4 N-RIC”) was named pBYKEAM-DNV1t4d.

Next, a RIC containing DENV 1 and 2 fused to the N-terminus and 3-4 fused to the C-terminus of the RIC antibody was constructed with a four-fragment ligation. pBYKEHM-h6D8-DENV114d was digested with SbfI-SpeI to obtain the vector, pBYKEHM-DV112 was digested SbfI-BsrGI to obtain a segment with DENV 1 and 2 fused to the heavy chain, pBYKEHM-h6D8-DENV1t4d was digested with BsrGI-BamHI to obtain the 3′ end of the heavy chain, and gBlock 1-2 was digested with BamHI-SpeI to obtain the linked DENV 3-4 antigens. The final construct (“DV 2H2”) was named pBYKEHM-DNV1t2-6D8-3t4d.

Four RICs, each containing a single DENV antigen (DENV-1, 2, 3, or 4) fused to the N-terminus of the RIC antibody were constructed by PCR-amplifying gBlock 1-2 and gBlock 3-4 with primers that end-tailored BsaI-SpeI restriction enzyme sites on each antigen. The BsaI-SpeI digested PCR fragments were then ligated into a pBYKEAM vector that contained an antibody heavy chain followed by an epitope tag. The resulting constructs were named pBYKEAM-DV1Hd, pBYKEAM-DV2Hd, pBYKEAM-DV3Hd, and pBYKEAM-DV4Hd (referred to as “DVx NRIC” with ‘x’ describing the antigen serotype).

Next, constructs contained the individual DENV antigens followed by a 6-histidine tag were created. The vector for all the constructs, pBYKEAM-BAGFPas6H, was similar to pBYe3R2K2Mc-BAZsE6H but had a pBYKEAM backbone and a GFP insert. The vector was digested XhoI-SpeI and the insert containing the antigen was obtained by a XhoI-SpeI digest of pBYKEAM-DV1Hd, pBYKEAM-DV2Hd, pBYKEAM-DV3Hd, or pBYKEAM-DV4Hd. The resulting constructs were named pBYKEAM-DV1-6H, pBYKEAM-DV2-6H, pBYKEAM-DV3-6H, and pBYKEAM-DV4-6H (referred to as “DVx-6H” with ‘x’ describing the antigen serotype).

A mouse RIC containing a single DENV-1 antigen fused to the murine IgG2a heavy chain N-terminus was created as follows. The constant region of the variable heavy chain of the murine 6D8 antibody was codon-optimized for expression in N. benthamiana and commercially synthesized (Integrated DNA Technologies). The synthesized gene was then ligated into an intermediate vector that contained murine 6D8 variable heavy chain as described in. The C-terminus of the heavy chain was linked to the 6D8 ‘d’ epitope tag to enable immune complex formation. To create the final N-terminal DENV-1 heavy chain fusion, the murine heavy chain segment was obtained with a BamHI-SacI digest of the intermediate heavy chain construct and ligated into the pBYKEAM-DV1Hd backbone. The resulting construct was named pBYKEAM-mIgG-DV1Hd. The murine light chain was cloned into a separate pBYKEAM plant expression vector. The constant region of the murine 6D8 light chain was commercially synthesized (Integrated DNA Technologies) and the variable region of the light chain was obtained from an intermediate vector. The final construct was named pBYKEAM-m6D8K_opt. Co-expression of the pBYKEAM-mIgG-DV1Hd and pBYKEAM-m6D8K_opt constructs resulted in the formation of a murine RIC containing DENV-1 on the antibody N-terminus (“DV1 mRIC”).

All the RICs in this example used the shortened ‘d’ epitope tag.

2.2 Agroinfiltration of Nicotiana benthamiana Plants

The BeYDV plant expression vectors for each construct described above were introduced into Agrobacterium tumefaciens EHA105 through electroporation. The resulting strains were confirmed by either restriction digestion or PCR and grown overnight at 30 C. The constructs were then used to infiltrate leaves of 5- to 6-week-old N. benthamiana plants maintained at 23-25 C. For the human and mouse RICs, vectors separately containing the DENV-fused 6D8 heavy chain and the light chain were co-infiltrated into N. benthamiana leaves. The plants used in this example have been silenced for xylosyltransferase and fucosyltransferase enzymes and produce a highly homogenous, human-like glycosylation pattern that can improve in vivo Fc receptor binding. The protocol for infiltration was conducted as described in (Diamos et al., “Codelivery of improved immune complex and virus-like particle vaccines containing Zika virus envelope domain III synergistically enhances immunogenicity,” Vaccine 38, 3455-3463 (2020)). The leaf samples were harvested 5 days post-infiltration (DPI) unless stated otherwise. Prior to harvest, the leaves were photographed for later analysis.

### 2.3 Protein Extraction and Purification

For experiments using crude leaf extracts, leaf samples of 100 mg were extracted in 500 ml of pH 8.0 extraction buffer containing 25 mM Tris-HCl, 125 mM NaCl, 3 mM EDTA, 0.1% Triton, 50 mM sodium ascorbate, and 2 mM PMSF. For the purification, the RIC leaf samples were homogenized in ice-cold, 1:2 w/V extraction buffer at pH 8.0 (25 mM Tris-HCl, 125 mM NaCl, 3 mM EDTA, 0.1% Triton, 50 mM sodium ascorbate, and 2 mM PMSF). Protein G column chromatography was used to purify the RIC proteins. The elutions were stored at −80° C. The 6-histidine tagged DENV antigens were purified with a metal affinity chromatography purification. Peak elutions were pooled, dialyzed against PBS, and stored at −80° C. for further analysis.

### 2.4 SDS-PAGE and Western Blot

The crude plant extracts and purified protein samples were assessed by SDS-PAGE gel and Western blots. The samples were mixed with SDS sample buffer (final concentration 50 mM Tris-HCl, pH 6.8, 2% SDS, 10% glycerol, 0.02% bromophenol blue) and loaded on 4-15% polyacrylamide gels (Bio-Rad). For reducing conditions, 300 mM DTT was added to the sample buffer and the samples were boiled for 10 min before being loaded on the gel. After the samples were run, the gels were either transferred to a PVDF membrane for a Western blot or stained with Coomassie stain (Bio-Rad, Hercules, CA, USA) following the manufacturer's instructions. For detection of the samples, the protein transferred membranes were blocked with 5% dry milk in PBST (PBS with 0.05% tween-20) overnight at 4° C., washed with PBST (3 washes, 5 min each), and probed with either a polyclonal anti-human IgG-horseradish peroxidase conjugate (Sigma), a polyclonal goat anti-mouse IgG-horseradish peroxidase conjugate (Sigma-Aldrich) or a mouse anti-poly-histidine antibody (Sigma).

### 2.5 C1q Binding and FcγRIII Binding

96-well medium-binding polystyrene plates (Thermo Fisher Scientific, Waltham, MA, USA) were coated with 15 μg/ml human complement C1q (PFA, MilliporeSigma, MA) in PBS for 1.5 hours at 37° C. The plates were washed 3 times with PBST, and then blocked with 5% dry milk in PBST for 30 minutes. After washing 3 times with PBST, purified human IgG and purified DV1-4 NRIC, DV1-4 CRIC, DV 2H2, DV1 NRIC, and DV1 mRIC fusions were added at 0.1 mg/ml with a 10-fold serial dilution and were incubated for 1.5 hours at 37° C. After washing 3 times with PBST, bound IgG was detected by incubating with a 1:500 dilution of an anti-human IgG (whole molecule) HRP-labeled probe (Sigma-Aldrich, St. Louis, MO, USA) for 1 hour at 37° C. The wells containing the DV1 mRIC were detected with a 1:250 dilution of a goat anti mouse IgG2A+HRP antibody (Southern Biotech, Birmingham, AL, USA). The plates were washed 4 times with PBST, developed with TMB substrate (Thermo Fisher Scientific, Waltham, MA, USA), stopped with 1M HCl, and the absorbance was read at 450 nm.

To test FcγRIII binding, a 96-well medium-binding polystyrene plate (Thermo Fisher Scientific, Waltham, MA, USA) was coated with 1.2 μg/ml human FcγRIIIA/CD16a protein (BioLegend, San Diego, CA, USA) in PBS for 1.5 hours at 37° C. The plates were washed 3 times with PBST and then blocked with 5% dry milk in PBST for 30 minutes. After washing 3 times with PBST, purified constructs were added at equimolar concentrations. The starting concentrations of DV1-4 NRIC, DV1-4 CRIC, and DV 2H2 were all at 24 μg/ml while the starting concentrations of DV1 NRIC and DV1 mRIC were at 17 μg/ml. These samples and a 10-fold dilution of each were applied to the wells and were incubated for 1.5 hours at 37° C. After washing 3 times with PBST, the bound constructs were detected by incubating the plate with a 1:500 dilution of an anti-human IgG (whole molecule) HRP-labeled probe (Sigma-Aldrich, St. Louis, MO, USA) for 1 hour at 37° C. The wells containing the DV1 mRIC were detected with a 1:250 dilution of a goat anti mouse IgG2A+HRP antibody (Southern Biotech, Birmingham, AL, USA) The plates were washed 4 times with PBST, developed with TMB substrate (Thermo Fisher Scientific, Waltham, MA, USA), stopped with 1M HCl, and the absorbance was read at 450 nm.

### 2.6 Immunization of Mice and Sample Collection

Groups of 6-7-week-old C57BL/6 mice (n=6) were immunized via subcutaneous injection with three doses that delivered an equal dose of 1 μg per DENV serotype. The constructs were first analyzed by SDS-PAGE to detect any cleavage products, then quantified by the ImageJ software and spectroscopy to determine the percentage of DENV-containing antigen. The groups were as follows: DV1-4 C-RIC, DV1-4 N-NRIC, DV 2H2, and the co-delivered four single antigen RICs (DV1 N-RIC, DV2 N-RIC, DV3 N-RIC, and DV4 N-RIC). Three mice were Immunized with PBS for a negative control. The doses were administered on days 0, 28, and 56. Serum was collected by submandibular bleed on days 0, 28, and 56, and 86. The spleens were collected on day 86 for further analysis. All animals were handled in accordance with the Animal Welfare Act and Arizona State University IACUC.

### 2.7 Spleen Cell Culture and IFN-γ Production

Spleens from immunized mice were mechanically dissociated into a single-cell suspension. The splenocytes were then resuspended in RPMI-C medium with 10% heat-inactivated FBS (Invitrogen, CA) to a cell concentration of 6.25×10⁶ cells/ml. After being aliquoted to 12-well tissue culture plates, the splenocytes were stimulated by being cultured in the presence of 10 μg/ml of each vaccine immunogen. Positive controls were prepared by adding the T-cell mitogen Con A (10 μg/ml, MilliporeSigma, MA) to control wells and negative control wells were prepared by adding the 6D8 antibody without any antigen. The cells were incubated for 48 hours in a 37° C. humidified Co₂ incubator. Then, the culture supernatants were collected and stored at −80° C. for cytokine production analysis.

### 3 Results

### 3.1 Protein Design and Expression

We and others have shown that displaying an antigen on RIC and other IgG fusions results in enhanced immunogenicity. Past work on improving the RIC platform has focused on altering the antigen fusion location from the antibody N-terminus to C-terminus and on modifying the epitope tag to enhance protein solubility. However, the variants of the RIC platform can only display a single antigen. Since pathogens can have multiple potential antigens or various serotypes, as in the case of Dengue virus, we focused on designing variants of the RIC platform that can display more than one antigen.

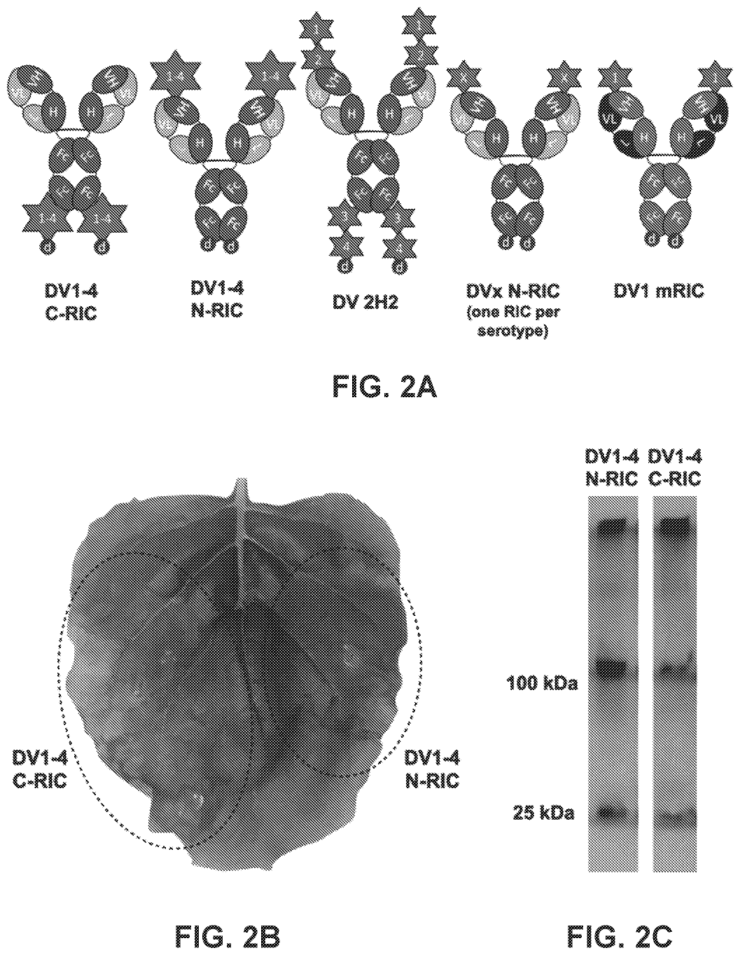

The constructs, as depicted in FIGS. 2A-2C: Construct depictions and expression analysis of DV1-4 RICs (A) Schematic depictions of the constructs used in this example. The stars indicate the serotype of the DV antigen while the circle symbolizes the shortened self-reactive antibody tag. The murine antibody shown as DV1 mRIC represents the different species of antibody. Error! Reference source not found. The DV1-4 C-RIC and DV1-4 N-RIC constructs have all four antigens tandemly linked to the antibody, while DV 2H2 has two antigens linked to the N-terminus and two antigens linked to the C-terminus of the humanized 6D8 IgG1 antibody heavy chain along with the 6D8 epitope tag to allow formation of immune complex. In addition, four DV N-RICs were created, each containing a single domain III DENV antigen from serotype 1, 2, 3, or 4. FIG. 2C showed bands at the expected sizes for a heavy chain fusion (95 kDa) and light chain (25 kDa). An ImageJ analysis of the heavy chain fusion band showed that the DV1-4 N-RIC sample had 28% more soluble protein than the DV1-4 C-RIC.

A similar analysis was conducted for the four individual DENV N-RICs. As shown in Error! Reference source not found. The four N-RICs were well-tolerated by the plants with little to no visible signs of necrosis at 5 DPI. These results were consistent across leaf samples that were harvested from two different leaves on the same infiltrated plant.

### 3.2 Protein Purification

After the RIC constructs were purified with protein G affinity chromatography, they were analyzed by SDS-PAGE gel electrophoresis followed by Coomassie staining. The gel results (Error! Reference source not found. The results showed that the constructs displayed the expected molecular mass under reducing conditions. Furthermore, the purified material was pure with little to no signs of contamination with native plant proteins. The gels were then analyzed with ImageJ to quantify the percentage of fully formed product. The individual, histidine tagged DENV antigens were purified with metal affinity chromatography and were similarly assessed.

### 3.3 Mouse Immunization Trial

The constructs were then tested in a mouse immunization study. Groups of C57BL/6 mice (n=6) were immunized subcutaneously with 4 μg of DENV domain III antigen (˜1 μg per serotype) delivered as DV1-4 N-RIC; DV1-4 C-RIC; DV 2H2; or co-delivered DV1, DV2, DV3, and DV4 N-RICs. Two other groups were added to compare the immunogenicity between a RIC with the previously tested 6D8 humanized antibody and a RIC with a mouse 6D8 antibody. DV1 was used as the antigen for both the RICs to enable a direct comparison. Sera samples were collected after each dose and stored at −80° C. for later testing to determine the endpoint antibody titers for each dose. In addition, spleens were collected at the conclusion of the example and used for a splenocyte antigen stimulation assay.

### 3.4 C1q and FcγRIII Binding Assay

The constructs were tested for binding to human C1q and FcγRIII receptors. As seen in Error! Reference source not found. The constructs bound to both the receptors. The RICs with four antigenic fusions all had similar binding while the DV1 hRIC had lower binding. The mRIC did not bind well, as expected, since it was being tested against human receptors.

### 4 Discussion

This example enhances prior research on the RIC platform and IgG fusions by testing the immunogenicity of a RIC that displays multiple antigens. IgG fusion strategies with single antigen fusions have repeatedly shown increased antigen immunogenicity upon fusion to a monomeric or polymeric antibody-based structure. Based on these findings, an IgG fusion platform that can display multiple antigens is of great use against pathogens, such as Dengue virus, malaria, and influenza, that have multiple potential antigens or serotypes. The strategy of including multiple antigens in a vaccine candidate has been successfully applied in the past with adenoviral vaccine vectors, hepatis B virus-like particles, tobacco mosaic virion conjugates, vesicular stomatitis virus-based delivery systems, antigen-fusions, and DNA vaccines. However, the use of IgG fusions to enhance the immunogenicity of multiple antigens has not been well-studied.

The constructs developed and tested in this example demonstrate the feasibility of creating fusion proteins with up to four antigens. These proteins were appropriately produced in the N. benthamiana plant expression system and showed evidence of proper assembly. Upon reduction, the constructs displayed molecular weight bands of the expected sizes. The variance between DV1-4 N-RIC and DV1-4 C-RIC in the level of soluble protein detected on a Western blot (Error! Reference source not found. FIG. 2C might be explained by the nature of the antigenic fusion position. The DV1-4 N-RIC, by virtue of having all four antigens linked to the N-terminus of the RIC antibody, will likely have hampered binding to the epitope tags on surrounding RIC fusions. In addition, the epitope tag used in this example has been shortened to limit the formation of large immune complexes that make the constructs insoluble. The combination of multiple antigen fusions near the antibody binding region and a short epitope tag with reduced antibody binding might make the DV1-4 N-RIC more soluble than the DV1-4 C-RIC. The design of the C-RIC does not place any inhibitions on the binding of the antibody to the epitope tag on surrounding RIC molecules. The antigens are linked to the antibody C-terminus and the epitope tag is placed after the last antigen. The increased potential for binding to and forming complexes could affect the overall solubility of the constructs. Future research in this area could focus on elucidating the importance of complex size on the immunogenicity of the RIC platform.

The C1q and FcγRIII binding data showed that the human RICs were able to bind the receptors as expected. The mRIC had weaker C1q binding and low FcγRIII since it was being tested against human receptors; however, the DV RICs with four antigens in various fusion strategies all had similar binding (Error! Reference source not found. Additional steps can include analyzing the mouse sera samples collected after each dose to determine the total endpoint titers against all four antigens and serotype-specific titers generated after the third dose. The Th1 response of the immunized C57BL/6 mice can also be assessed by measuring the level of IgG2c antibodies and IFN-γ. In addition, a viral plaque reduction neutralization assay for all four DENV serotypes can be conducted to determine whether the vaccine constructs produced neutralizing antibodies.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, and/or computer readable media or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

What is claimed is:

1. A vaccination composition, the composition comprising: a multivalent recombinant immune complex (RIC), wherein the RIC comprises an immunoglobulin heavy chain; at least one epitope tag, wherein the immunoglobulin heavy chain binds the epitope tag; and at least fragments of antigens from at least two viral serotypes.

2. The vaccination composition of claim 1, wherein the antigens are from at least three viral serotypes, at least four viral serotypes, at least five viral serotypes, at least six viral serotypes, or more viral serotypes.

3. The vaccination composition of claim 1, wherein the antigens comprise a dengue virus (DENV) domain III antigen from four DENV serotypes.

4. The vaccination composition of claim 1, wherein the epitope tag is linked to the antigens.

5. The vaccination composition of claim 1, wherein the epitope tag is linked to the C-terminus and/or the N-terminus of the immunoglobulin heavy chain.

6. The vaccination composition of claim 1, wherein one or more of the antigens are linked to the C-terminus and/or the N-terminus of the immunoglobulin heavy chain.

7. The vaccination composition of claim 1, further comprising an immunoglobulin light chain.

8. The vaccination composition of claim 1, comprising an Immunoglobulin G (IgG).

9. The vaccination composition of claim 1, comprising a human or humanized IgG.

10. A method of generating an immune response against a virus in a mammalian subject, the method comprising: administering to the mammalian subject a multivalent recombinant immune complex (RIC) that comprises: an immunoglobulin heavy chain; at least one epitope tag, wherein the immunoglobulin heavy chain binds the epitope tag; and at least fragments of antigens from at two viral serotypes.

11. The method of claim 10, wherein the virus comprises a dengue virus (DENV) and wherein the antigens comprise a DENV domain III antigen from four DENV serotypes.

12. The method of claim 10, wherein the antigens comprise a dengue virus (DENV) domain III antigen from four DENV serotypes.

13. The method of claim 10, wherein the epitope tag is linked to the antigens.

14. The method of claim 10, wherein the epitope tag is linked to the C-terminus and/or the N-terminus of the immunoglobulin heavy chain.

15. The method of claim 10, wherein one or more of the antigens are linked to the C-terminus and/or the N-terminus of the immunoglobulin heavy chain.

16. The method of claim 10, further comprising an immunoglobulin light chain.

17. The method of claim 10, comprising an Immunoglobulin G (IgG).

18. The method of claim 17, wherein the IgG comprises a human or humanized IgG.

19. A recombinant vector comprising a nucleic acid molecule that encodes a recombinant immune complex (RIC), wherein the RIC comprises an immunoglobulin heavy chain; at least one epitope tag, wherein the immunoglobulin heavy chain binds the epitope tag; and at least fragments of antigens from at least two viral serotypes.

20. The recombinant vector of claim 19, wherein a plasmid comprises the recombinant vector.


SEQUENCE LISTING (28 sequences)

SEQ ID NO: 1 - DNA, 57 bp, synthetic construct
  misc_feature 1..57: note = Synthetic: IgG heavy chain signal
  source 1..57: mol_type = other DNA
atgggatggtcttgcatcatactctttcttgttgcaactgctacaggtgtccactct

SEQ ID NO: 2 - DNA, 1338 bp, synthetic construct
  misc_feature 1..1338: note = Synthetic: IgG1 heavy chain
  source 1..1338: mol_type = other DNA
gatgttcagcttctcgagtctggaggtggtcttgtgcaacctggaggttccttgagactctcctgtgcagcttcagggtttgacttcagtaggtactggatgagttgggttcgtcaagctcctgggaaaggactagaatggattggagagatcaatccagattcaagtaccatcaactatactccatctctgaaggatcgcttcaccatttccagagacaatgccaagaacacgttgtatcttcagatgaacagcttgaggactgaagacacagccttgtactactgcacaagacagggctatggctacaactactggggtcaaggcaccactgtcacagtgtcttcagctagcaccaaaggtccatcggtctttccactggcaccttcttccaagagtacttctggaggcacagctgcactgggttgtcttgtcaaggactactttccagaacctgttacggtttcgtggaactcaggtgctctgaccagtggagtgcacacctttccagctgttcttcagtcctcaggattgtattctcttagcagtgttgtgactgttccatcctcaagcttgggcactcagacctacatctgcaatgtgaatcacaaacccagcaacaccaaggttgacaagaaagttgagcccaagtcttgtgacaagactcatacgtgtccaccgtgcccagcacctgaacttcttggaggaccgtcagtcttcttgtttcctccaaagcctaaggataccttgatgatctccaggactcctgaagtcacatgtgtagttgtggatgtgagccatgaagatcctgaggtgaagttcaactggtatgtggatggtgtggaagtgcacaatgccaagacaaagccgagagaggaacagtacaacagcacgtacagggttgtctcagttctcactgttctccatcaagattggttgaatggcaaagagtacaagtgcaaggtctccaacaaagccctcccagcccccattgagaagaccatttccaaagcgaaagggcaaccccgtgaaccacaagtgtacacacttcctccatctcgcgatgaactgaccaagaaccaggtcagcttgacttgcctggtgaaaggcttctatccctctgacatagctgtagagtgggagagcaatgggcaaccggagaacaactacaagactacacctcccgttctcgattctgacggctccttcttcctctacagcaagctcacagtggacaagagcaggtggcaacaagggaatgtcttctcatgctccgtgatgcatgaggctcttcacaatcactacacacagaagagtctctccttgtctccgggtaaa

SEQ ID NO: 3 - DNA, 45 bp, synthetic construct
  misc_feature 1..45: note = Synthetic: Linker
  source 1..45: mol_type = other DNA
ggaggtggcggatcaggtggaggcggttcaggcggaggtggatcc

SEQ ID NO: 4 - DNA, 300 bp, synthetic construct
  misc_feature 1..300: note = Synthetic: DENV1 EdIII
  source 1..300: mol_type = other DNA
tcttacgttatgtgcactggatcttttaagcttgagaaagaggtggccgaaacccaacacggtactgttttggttcaagttaagtacgagggtaccgacgcaccatgcaaaatccccttctcttctcaagatgagaagggagttactcagaacggtagacttatcaccgcaaatcctatagttacagataaggagaaacctgtgaatatagaggccgagcctccgttcggggagtcttatatcgttgtcggcgctggggagaaggcgcttaaattgtcatggttcaagaaggggtcatca

SEQ ID NO: 5 - DNA, 15 bp, synthetic construct
  misc_feature 1..15: note = Synthetic: Linker (1-2)
  source 1..15: mol_type = other DNA
ggaggttcaggttcc

SEQ ID NO: 6 - DNA, 300 bp, synthetic construct
  misc_feature 1..300: note = Synthetic: DENV2 EdIII
  source 1..300: mol_type = other DNA
tcttactctatgtgcactgggaaattcaaagtcgttaaggagatagctgaaactcagcacggaaccatcgttattagagttcagtacgaaggagatggatctccatgcaaaaccccttttgaaatcatggaccttgagaagagacacgttttgggtagacttactactgtgaacccgatcgttactgagaaggattccccagtgaacatagaagctgagcctccgttcggtgactcttacatcatcatcggtgttgagcctggccagctcaagttagactggtttaagaagggttcatcc

SEQ ID NO: 7 - DNA, 15 bp, synthetic construct
  misc_feature 1..15: note = Synthetic: Linker (2-3)
  source 1..15: mol_type = other DNA
ggaggttcaggttct

SEQ ID NO: 8 - DNA, 300 bp, synthetic construct
  misc_feature 1..300: note = Synthetic: DENV3 EdIII
  source 1..300: mol_type = other DNA
tcttacgctatgtgtttgaataccttcgttttgaagaaagaagtttctgaaacccagcacggaaccatccttattaaggttgagtacaagggagaagacgctccatgcaagatccctttctctactgaggatggtcaaggcaaggcccacaacgggagacttatcactgcaaacccagtcgttaccaagaaagaagagccagtaaacatcgaggctgaaccaccctttggagagtccaacatagtgatcggtatcggagacaaggctcttaagatcaattggtatcgtaaaggctcatca

SEQ ID NO: 9 - DNA, 15 bp, synthetic construct
  misc_feature 1..15: note = Synthetic: Linker (3-4)
  source 1..15: mol_type = other DNA
ggaggttcaggatct

SEQ ID NO: 10 - DNA, 297 bp, synthetic construct
  misc_feature 1..297: note = Synthetic: DENV4 EdIII
  source 1..297: mol_type = other DNA
tcttacactatgtgttctggaaagttttctatcgataaggagatggctgagactcagcacggtacaactgttgttaaggttaagtacgaaggagccggagccccatgcaaagttccaatcgagattagagacgtcaacaaagagaaggtcgttggaaggatcatctctcccaccccatttgctgaaaacaccaactctgttacaaacatcgaattggaaagacctctcgactcctatatcgttatcggagttggcgattctgctcttacccttcactggttcagaaagggatcttct

SEQ ID NO: 11 - skipped

SEQ ID NO: 12 - DNA, 18 bp, synthetic construct
  misc_feature 1..18: note = Synthetic: Epitope tag d
  source 1..18: mol_type = other DNA
tacaagctggacatatct

SEQ ID NO: 13 - DNA, 57 bp, synthetic construct
  misc_feature 1..57: note = Synthetic: IgG light chain
  source 1..57: mol_type = other DNA
atgggatggtcttgcatcattctcttcttggtagccacagctacaggtgtccactcc

SEQ ID NO: 14 - DNA, 654 bp, synthetic construct
  misc_feature 1..654: note = Synthetic: IgG light chain kappa
  source 1..654: mol_type = other DNA
gatgttttgatgactcaaagccctctctcacttcctgtgactcttggacagcccgcatccatatcttgcagatctagtcagagtattgttcatagtaacggcaacacctacttggaatggtatctgcagaaaccaggccagtctccaaagcttctgatctacaaggcttccaatcgtttctctggtgtcccagacaggtttagtggcagtggatcagggactgacttcacattgaagatcagcagagttgaggctgaagatgcgggagtgtactattgtcttcaaggttcacatgttccgtcaacgtttggaggtgggaccaaagtggagatcaagactgttgcggcgccatctgtcttcatctttcctccatctgatgaacaactcaagtctggaactgcttctgttgtgtgccttctgaacaacttctatcctagagaagccaaagtacagtggaaggttgacaatgctcttcaatcaggtaactcccaggagagtgtcacagagcaagattccaaggattccacctacagcctctcaagtaccttgacgttgagcaaggcagactatgagaaacacaaagtgtacgcatgcgaagtcactcatcagggcctgtcatcacccgtgacaaagagcttcaacaggggagagtgt

SEQ ID NO: 15 - DNA, 72 bp, synthetic construct
  misc_feature 1..72: note = Synthetic: Barley alpha amylase signal peptide
  source 1..72: mol_type = other DNA
atggctaacaagcacctctcattgtctctcttccttgtgctccttggtctttctgcttctcttgcttctggt

SEQ ID NO: 16 - protein, 19 aa, synthetic construct
  REGION 1..19: note = Synthetic: IgG heavy chain signal peptide
MGWSCIILFLVATATGVHS

SEQ ID NO: 17 - protein, 446 aa, synthetic construct
  REGION 1..446: note = Synthetic: IgG heavy chain
DVQLLESGGGLVQPGGSLRLSCAASGFDFSRYWMSWVRQAPGKGLEWIGEINPDSSTINYTPSLKDRFTISRDNAKNTLYLQMNSLRTEDTALYYCTRQGYGYNYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 15 aa, synthetic construct
  REGION 1..15: note = Synthetic: Linker
GGGGSGGGGSGGGGS

SEQ ID NO: 19 - protein, 100 aa, synthetic construct
  REGION 1..100: note = Synthetic: DENV1 EdIII
SYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSS

SEQ ID NO: 20 - protein, 5 aa, synthetic construct
  REGION 1..5: note = Synthetic: Linker (1-2, 2-3, 3-4)
GGSGS

SEQ ID NO: 21 - protein, 100 aa, synthetic construct
  REGION 1..100: note = Synthetic: DENV2 EdIII
SYSMCTGKFKVVKEIAETQHGTIVIRVQYEGDGSPCKTPFEIMDLEKRHVLGRLTTVNPIVTEKDSPVNIEAEPPFGDSYIIIGVEPGQLKLDWFKKGSS

SEQ ID NO: 22 - protein, 100 aa, synthetic construct
  REGION 1..100: note = Synthetic: DENV3 EdIII
SYAMCLNTFVLKKEVSETQHGTILIKVEYKGEDAPCKIPFSTEDGQGKAHNGRLITANPVVTKKEEPVNIEAEPPFGESNIVIGIGDKALKINWYRKGSS

SEQ ID NO: 23 - protein, 99 aa, synthetic construct
  REGION 1..99: note = Synthetic: DENV4 EdIII
SYTMCSGKFSIDKEMAETQHGTTVVKVKYEGAGAPCKVPIEIRDVNKEKVVGRIISPTPFAENTNSVTNIELERPLDSYIVIGVGDSALTLHWFRKGSS

SEQ ID NO: 24 - skipped

SEQ ID NO: 25 - protein, 6 aa, synthetic construct
  REGION 1..6: note = Synthetic: Epitope tag d
YKLDIS

SEQ ID NO: 26 - protein, 19 aa, synthetic construct
  REGION 1..19: note = Synthetic: IgG light chain signal peptide
MGWSCIILFLVATATGVHS

SEQ ID NO: 27 - protein, 218 aa, synthetic construct
  REGION 1..218: note = Synthetic: IgG light chain kappa
DVLMTQSPLSLPVTLGQPASISCRSSQSIVHSNGNTYLEWYLQKPGQSPKLLIYKASNRFSGVPDRFSGSGSGTDFTLKISRVEAEDAGVYYCLQGSHVPSTFGGGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 24 aa, synthetic construct
  REGION 1..24: note = Synthetic: Barley alpha amylase signal peptide
MANKHLSLSLFLVLLGLSASLASG